(19) United States
(12) Patent Application Publication
(10) Pub. No.: US 2026/0035671 A1
(43) Pub. Date: Feb. 5, 2026

(54) COMPOSITIONS COMPRISING PREVASCULARIZED ORGANOIDS AND METHODS FOR ENHANCING ANGIOGENESIS

(71) Applicants: The Trustees of Columbia University in the City of New York, New York, NY (US); Cedars-Sinai Medical Center, Los Angeles, CA (US)

(72) Inventors: Samuel K. SIA, New York, NY (US); Darragh KENNEDY, New York, NY (US); Mary Katharyn FATEHI, New York, NY (US); Dhruv SAREEN, Los Angeles, CA (US)

(21) Appl. No.: 19/288,789

(22) Filed: Aug. 1, 2025

Related U.S. Application Data
(60) Provisional application No. 63/678,130, filed on Aug. 1, 2024.

(51) Int. Cl. C12N 5/071; A61K 9/00; A61K 35/44; A61P 9/10; C12N 5/00

(52) U.S. Cl. CPC C12N 5/0692; A61K 9/0019; A61K 35/44; A61P 9/10; C12N 5/0062; C12N 2513/00; C12N 2533/74

(57) ABSTRACT

Methods for producing prevascularized organoids, and compositions comprising such prevascularized organoids, are provided. Methods of treating ischemic conditions and promoting angiogenesis by administering the prevascularized organoids to a subject in need thereof are also provided.

### CROSS-REFERENCE TO RELATED APPLICATIONS

This application claims benefit of U.S. Provisional Application No. 63/678,130, filed Aug. 1, 2024, the contents of which are hereby incorporated by reference.

### STATEMENT OF GOVERNMENT SUPPORT

This invention was made with government support under grant number HL141935 awarded by the National Institutes of Health. The government has certain rights in the invention.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

Vascular diseases, including peripheral artery disease can lead to ischemia, which in turn causes life-threatening outcomes such as amputation or myocardial infarction (1)(2). To treat ischemia, therapeutic angiogenesis aims to increase blood flow to ischemic tissue (3), including cell-based therapies such as endothelial cells and mesenchymal stem cells due to their ability to secrete potent angiogenic proteins and cytokines (3,4). Moreover, compared to suspensions of cells, prevascularized organoids may improve retention of cells at the site of ischemia as well as promote rapid vascularization (5)(6)(7). In addition to studies of MSCs for treatment of hindlimb ischemia (e.g. through secretion of pro-angiogenic paracrine factors), endothelial cells (ECs) have also been efficacious for treatment of hindlimb ischemia, including HUVECs in a hyaluronan gel (8), co-administered with smooth muscle cells (9) and alone as iPSC-derived ECs (10). Differentiated ECs showed improvement in hindlimb ischemia outcomes and displayed higher retention and integration into host vasculature in ischemic limbs, compared to MSCs in vivo (11).

Organoids or spheroids can present cells with microenvironments that improve cell viability and promote angiogenic potential (e.g. via increased secretion of proangiogenic factors, or through formation of capillary-like structures that can rapidly anastomose with host vasculature). For example, some studies showed that organoids composed of MSCs and ECs or endothelial colony forming cells (ECFCs) exhibited high cell viability in vivo, high cytokine release and high angiogenesis in mouse models of hindlimb ischemia and ischemic stroke (12) (13). Nevertheless, the clinical translation of organoids towards therapy has been limited by several challenges.

First, previous organoids often used cells which present immunogenic effects. For example, human primary cells such as human umbilical vein endothelial cells (HUVECs) presented potential immunogenic responses (14). Isolation of ECs from patients use methods of low yield, thus hampering their clinical potential (15). Some isolation methods, such as those used from brain tissue, are vulnerable to contamination from other cell types such as fibroblasts, are time-consuming and inefficient (16). On the other hand, human iPSC-derived stem cells (hiPSCs) may be non-immunogenic either with HLA-matched allogeneic source or autologous source (17) (18), but their differentiation can be expensive and span multiple weeks and efficiency is typically limited to 60 to 70% (19-22). There has been a recent trend towards the use of universal hypoimmune iPSCs for scalable allogeneic cell replacement, thereby avoiding immunogenicity of transplanted cells (23-26). Moreover, there have been few demonstrations with Good Manufacturing Practice (GMP)-compliant processes for generating and differentiating human iPSCs (27) (28), while important for clinical translation to ensure safety, quality, efficacy and fulfillment of regulatory requirements (29).

As a second challenge, production methods of organoids have been low throughput or harsh. Previous approaches include culturing cells in spinner flasks (30), hanging drops (31) and centrifugation (32), but these approaches offer little control over sizes of organoids, are difficult to continuously observe to enforce biomanufacturing standards, and may impair cell function or survival due to harsh handling. While some methods to generate human iPSC-derived blood vessel organoids have been published, they were low-throughput (˜100 organoids per well plate) (33).

Third, previous techniques for generating organoids used reagents that were not GMP-compatible, and therefore ill-suited for clinical translation. To aid maturation and stabilization of vascular networks, some previous studies used Matrigel (31) (34), which is chemically ill-defined and has heterogeneous mechanical properties (35), exhibits lot-to-lot variations, and its mouse cell origin makes human clinical translation very challenging (36).

Fourth, applicability of therapeutic organoids would be enhanced if the organoids could be injected easily (similar to injectable hydrogel materials) into a target area (31).

The present invention addresses these challenges and provides methods of producing prevascularized organoids, including for use in promoting angiogenesis and treatment of ischemic conditions.

### SUMMARY OF THE INVENTION

Provided herein a method for producing prevascularized organoids, the method comprising providing a sacrificial hydrogel scaffold, seeding a population of cells comprising endothelial cells into the sacrificial hydrogel scaffold, culturing the population of cells to form prevascularized organoids, and removing the sacrificial hydrogel scaffold to release the prevascularized organoids.

Additionally, an in vitro generated three-dimensional prevascularized organoid derived from a composition of cells comprising human iECs is provided.

Also provided herein is a method of treating a subject having, or at risk of having, an ischemic condition, the method comprising administering to the subject an amount of a composition comprising a prevascularized organoid.

A method of promoting angiogenesis in a subject is also provided, the method comprising administering to the subject an amount of a composition comprising a prevascularized organoid.

### BRIEF DESCRIPTION OF THE DRAWINGS

FIGS. 1A-1B: Schematic diagram of the therapeutic organoid method for producing iEC-containing organoids. FIG. 1A) MSCs and iECs are combined after culturing each cell type. Then, the cells are seeded into the sacrificial hydrogel scaffold. Over time, the cells migrate and form organoids with a core-shell structure in the presence of maintenance media. FIG. 1B) Within the core, sprouting occurs with the introduction of vasculogenic media. Organoids are then added to the chick chorioallantoic membrane (CAM), demonstrating enhanced angiogenesis.

FIGS. 2A-2F: Characterization of iECs and organoids of varying cell combinations, using the sacrificial release method. FIG. 2A) Immunocytochemistry staining for (i) CD31 and VEGFR2, (ii) CD31 and CD144, (iii) CD31 and vWF for Day 21 iECs. CD31 is labeled in red, DAPI is labeled in blue, and all other markers are labeled in green. FIG. 2B) LDL assay for Day 22 iECs. FIG. 2C) Flow cytometry analysis of Day 25 iECs for markers CD31 and CD144 expression. Cell numbers at varying fluorescence intensities for FITC-A (bottom left) and APC-A (bottom right). The control sample is in red and the experimental sample is in blue. FIG. 2D) Organoid progression over the course of 8 days for iECs only (top row) and the 1:1 iEC:MSC ratio (bottom row). The day 8 image of the 1:1 ratio is enlarged (right), where arrows indicate sprouting. Scale bar is 200 μm. FIG. 2E) Average organoid diameters for each cell ratio over 8 days (n≥20 organoids), where 1:9, 1:3, and 1:1 indicate iEC:MSC ratio. FIG. 2F) Average number of sprouts and sprout lengths for each organoid cell ratio (n˜45 organoids). **** indicates p<0.0001.

FIGS. 3A-3D: In vitro studies on the effect of the secretome of EC:MSC cell combinations and organoids on angiogenesis. FIG. 3A) Visual representation of the migration of iECs when cultured in PBS, single cell-conditioned, or organoid-conditioned media. FIG. 3B) Quantification of gap closure due to iEC migration, n=3-5 per group (** denotes p<0.01, ns denotes non-significant) (related to FIG. 3A). FIG. 3C) Tube formation assay showing the influence of conditioned media on iEC angiogenesis. White arrows point to junctions. Scale bar=400 μm. FIG. 3D) Quantification of iEC tube formation represented by average number of junctions per field, n=3 per group (** denotes p<0.005, *** denotes p<0.001, ns denotes non-significant) (related to FIG. 3C). All data are mean±standard deviation.

FIGS. 4A-4E: In vivo studies of angiogenic potential of the organoid therapy in the chick chorion allantois membrane (CAM) assay. FIG. 4A) Schematic describing the methodology and timeline of the CAM assay. FIG. 4B) Images of organoid, single-cell combination, and PBS therapy on the CAM, with indications of angiogenic parameters, junctions and endpoints, indicated. Angiogenic effects of PBS vs. single-cell combination vs. organoid (n=3 per group), expressed by (c) % increase in number of junctions, (d) % increase in number of endpoints, and (e) % increase in vessel length. * denotes p<0.05, ** denotes p<0.01, *** p<0.005. All data are mean±standard deviation.

FIG. 5: In vivo studies of the fate and angiogenic potential of the organoid therapy in the mouse hindlimb. Left) Schematic describing the therapeutic groups and mouse hindlimb ischemia model. Right) Schematic describing the tests performed to study 1) organoid/single-cell therapy retention (fluorescent measurement) and 2) effects on the muscle (histology on gastrocnemius muscle).

FIG. 6: Retention of cells or organoids in the hindlimb. Fluorescence was measured in radiance using an IVIS machine. Whole-body fluorescent imaging of fluorescent-tagged cells and organoids in the mouse hindlimb.

FIG. 7: Tarlov score in mice seven (7) days after treatment with saline, organoids and single-cell suspensions for hindlimb ischemia.

FIGS. 8A-8B: Regenerating fibers in the gastrocnemius muscle 28 days after both injury induction and injection with saline, single-cell suspensions, or organoids. FIG. 8A shows representative images. Scale bar=20 μm. FIG. 8B is a quantification of regenerating fibers as a percentage of all fibers.

FIGS. 9A-9B: Image of prevascularized organoids pre-injection and post-injection. FIG. 9A: Image of prevascularized organoid pre-injection. FIG. 9B: Image of prevascularized organoid post-injection with a 25-gauge needle.

### DETAILED DESCRIPTION OF THE INVENTION

Provided herein a method for producing prevascularized organoids, the method comprising providing a sacrificial hydrogel scaffold, seeding a population of cells comprising endothelial cells into the sacrificial hydrogel scaffold, culturing the population of cells to form prevascularized organoids, and removing the sacrificial hydrogel scaffold to release the prevascularized organoids.

In some embodiments, the sacrificial hydrogel scaffold is formed by placing a hydrogel onto a mold containing at least one scaffold template, preferably wherein the mold contains up to 500, up to 250, up to 100, up to 50, or up to 10 scaffold templates. Thus, a single mold may be used to produce multiple scaffolds for high-throughput prevascularized organoid production.

In some embodiments, the mold comprises polydimethylsiloxane (PDMS), preferably wherein the mold is formed by casting the PDMS into an SU-8 master mold.

In some embodiments, the hydrogel comprises cellulose, chitosan, alginate, hyaluronic acid, collagen, gelatin, poly(vinyl alcohol), polyethylene glycol, and/or polyacrylamide.

In some embodiments, the hydrogel comprises an alginate, preferably 1-5% alginate, more preferably about 3% alginate.

In some embodiments, the hydrogel lacks a solubilized basement membrane preparation extracted from the Engelbreth-Holm-Swarm (EHS) sarcoma cells or lacks any Matrigel.

In some embodiments, the sacrificial hydrogel scaffold is crosslinked, preferably by exposure to CaCl₂, more preferably by exposure to about 60 mM CaCl₂.

In some embodiments, the crosslinked sacrificial hydrogel scaffold is stored in about 1.8 mM CaCl₂ prior to cell seeding.

In some embodiments, the crosslinked sacrificial hydrogel is exposed to neutralization media, preferably Endothelial Cell Basal Medium MV2 supplemented with 10% serum, preferably wherein the serum is fetal bovine serum (FBS) or human AB serum, when the population of cells is about to be seeded. Any basal cell medium appropriate for endothelial cell culture is contemplated for use as a neutralization media or culturing cells, e.g., iECs. In some embodiments, the basal cell medium may be supplemented with additional ingredients (e.g., serum) to create a growth medium e.g., GMPLV2 medium.

In some embodiments, about 1×10⁵ cells to about 5×10⁶ cells, preferably about 6×10⁵ cells, are seeded into the sacrificial hydrogel scaffold. The number of cells seeded into the scaffold may be adjusted to produce the desired about of prevascularized organoids.

In some embodiments, the sacrificial hydrogel scaffold comprises a plurality of microwells, wherein each microwell of the plurality is greater than 150 μm in diameter, preferably about 200 μm in diameter. In some embodiments, each microwell is 200, 225, 250, 300, 400, or 500 μm or greater in diameter.

In some embodiments, the sacrificial hydrogel scaffold comprises 500-2,000 microwells, preferably 1,000-1,500 microwells, more preferably about 1,300 microwells. The number of microwells in the scaffold may be adjusted to produce the desired about of prevascularized organoids.

In some embodiments, the population of cells comprising endothelial cells comprises vascular endothelial cells, induced pluripotent stem cell (iPSC)-derived vascular endothelial cells (iECs), and/or mesenchymal stem cells (MSCs).

In some embodiments, the population of cells comprising endothelial cells comprises iECs, and the iECs express CD31, CD144, VEGFR2, and vWF, preferably wherein greater than 90% of the iECs express CD31 and CD144.

In some embodiments, the population of cells comprising endothelial cells comprises iECs and MSCs in a 1:1 to 1:9 iEC:MSC ratio, preferably a 1:1 to 1:3 iEC:MSC ratio, more preferably in an about 1:1 iEC:MSC ratio.

In some embodiments, the population of cells comprises Day 17 iECs and/or Passage 5 to Passage 8 (P5-P8) MSCs.

In some embodiments, the population of cells comprises human cells.

In some embodiments, the population of cells is derived from an HLA-matched allogeneic source or an autologous source.

In some embodiments, the cells are cultured in Endothelial Cell Basal Medium MV2 supplemented with 10% serum after seeding, preferably about three to four days after seeding, preferably wherein the serum is fetal bovine serum (FBS) or human AB serum.

In some embodiments, Endothelial Cell Growth Medium LV2 media is introduced on the fourth day of cell culturing. Endothelial Cell (EC) Growth Medium LV2 medium (also referred to as “GMPLV2 medium”) which is Endothelial Cell Basal Medium MV2 (PromoCell), or another basal cell medium appropriate for endothelial cell culture, supplemented with VEGF₁₆₅ (260 ng/ml), fetal bovine serum or human AB serum (0.05 mL/mL), hydrocortisone (0.2 μg/mL), recombinant human FGF2-basic (153 a.a.) (10 ng/ml), animal-free recombinant human EGF, 6.2 kDa (5 ng/mL), L-ascorbic acid (1 μg/mL) and IGF-I LR3 (20 ng/ml).

In some embodiments, the population of cells are cultured for 4 to 12 days, preferably about 8 days, after seeding the cells into the sacrificial hydrogel scaffold.

In some embodiments, the sacrificial hydrogel scaffold is removed by exposing the sacrificial hydrogel scaffold to a chelator, preferably wherein the chelator is sodium citrate, preferably about 5% sodium citrate.

In some embodiments, the released prevascularized organoids comprise prevascularized organoids of about 120-160 μm in diameter, preferably about 140 μm in diameter.

In some embodiments, the released prevascularized organoids comprise about 1 to 2 vascular sprouts on average, preferably about 1.4 vascular sprouts on average, and/or comprise an average vascular sprout length of about 100-150 μm.

In some embodiments, the released prevascularized organoids are injectable through a 25-gauge needle or a larger needle, preferably wherein the injected organoids maintain their structural integrity and/or diameter upon injection. A 27-gauge needle or 30-gauge needle may also be used to inject the organoids.

In some embodiments, the organoids, secretions from the organoids, and/or medium conditioned with the organoids promote endothelial cell migration and/or angiogenesis in vitro, and/or promote endothelial cell migration, angiogenesis, blood flow, tissue regeneration, and/or limb function in vivo.

In some embodiments, the organoids, secretions from the organoids, and/or medium conditioned with the organoids promote endothelial cell migration and/or angiogenesis in vitro, and/or promote endothelial cell migration, angiogenesis, blood flow, tissue regeneration, and/or limb function in vivo.

In some embodiments, the method produces about 500-10,000 organoids per scaffold, preferably 1,000-2,000 organoids per scaffold, more preferably about 1,300 organoids per scaffold. The scaffold specifications, e.g., number of miniwells within the scaffold or scaffold size, may be adjusted to produce the number of desired prevascularized organoids.

In some embodiments, the method is Good Manufacturing Practices (GMP)-compliant and/or GMP-compatible.

Also provided herein is an in vitro generated three-dimensional prevascularized organoid derived from a composition of cells comprising human iECs.

In some embodiments, the iECs express CD31, CD144, VEGFR2, and vWF, preferably wherein greater than 90% of the iECs express CD31 and CD144.

In some embodiments, the composition of cells further comprises MSCs.

In some embodiments, the composition of cells comprises Day 17 iECs and/or P5 to P8 MSCs.

In some embodiments, the composition of cells comprises iECs and MSCs in a 1:1 to 1:9 iEC:MSC ratio, preferably a 1:1 to 1:3 iEC:MSC ratio, more preferably in an about 1:1 iEC:MSC ratio.

In some embodiments, the organoid is about 120-160 μm in diameter, preferably about 140 μm in diameter.

In some embodiments, the organoid comprises about 1 to 2 vascular sprouts, and/or comprises a vascular sprout about 100-150 μm in length.

In some embodiments, the human iECs are derived from an HLA-matched allogeneic source or an autologous source.

Also provided is a composition comprising a plurality of the prevascularized organoids described herein, the plurality comprising between about 1,000 to 250,000 organoids, preferably about 75,000 to 100,000 organoids. The number of prevascularized organoids in the composition may be adjusted to a desired amount., for example, a therapeutic amount.

Also provided is a composition comprising a plurality of the prevascularized organoids described herein, wherein the plurality of prevascularized organoids is generated from an isolated population of about 1×10⁵ to 10×10⁶ cells, preferably about 1×10⁵ to 1×10⁶ cells, preferably about 5×10⁵ to 7×10⁷ cells, preferably about 600,000 cells. The number of cells used to generate the prevascularized organoids may be adjusted to provide the desired number of organoids.

Also provided is a composition comprising the plurality of the prevascularized organoids described herein, the plurality comprising about 1×10⁶ to 100×10⁶ cells in organoids, preferably about 10×10⁶ to 50×10⁶ cells in organoids. The number of total cells in organoids in the composition may be adjusted as desired, and may be accomplished, for example, by adjusting the number of organoids in the composition.

Also provided is pharmaceutical composition comprising the plurality of prevascularized organoids described herein, and a physiologically acceptable or pharmaceutically acceptable carrier.

Also provided is a pharmaceutical composition comprising the prevascularized organoids produced by any one of the methods described herein, and a physiologically acceptable or pharmaceutically acceptable carrier.

Also provided is a method of treating a subject having, or at risk of having, an ischemic condition, the method comprising administering to the subject an amount of any one of the compositions or the pharmaceutical compositions described herein.

In some embodiments, the ischemic condition is critical limb-threatening ischemia (CLTI).

In some embodiments, the subject has or is at risk of having a vascular disease.

In some embodiments, the vascular disease is peripheral artery disease (PAD).

In some embodiments, blood flow is increased to an ischemic tissue of the subject.

Also provided is a method of promoting angiogenesis in a subject, the method comprising administering to the subject an amount of any one of the compositions or the pharmaceutical compositions described herein.

In some embodiments, the subject has or is at risk of having an ischemic condition and/or critical threatening limb ischemia (CTLI).

In some embodiments, the subject has or is at risk of having a vascular disease.

In some embodiments, the vascular disease is peripheral artery disease (PAD).

In some embodiments, blood flow is increased to an ischemic tissue of the subject.

In some embodiments, the composition or pharmaceutical composition is administered intramuscularly.

In some embodiments, the composition or pharmaceutical composition is administered into or proximal to ischemic tissue.

In some embodiments, the composition or pharmaceutical composition is injected through a needle diameter greater than or equal to a 25-gauge needle. Needles as small as a 30-gauge needle may also be used.

In some embodiments, about 10,000 to 200,000 organoids, preferably about 1×10⁵ organoids, are administered to the subject.

In some embodiments, the administration is an HLA-matched allogeneic administration or an autologous administration.

In some embodiments, the subject displays increased angiogenesis, blood flow, tissue regeneration, and/or limb function at the site of administration. In some embodiments, these affects are detected within 14 days of administration.

Also provided is a composition comprising the prevascularized organoids produced by the method described herein prior to their release from the scaffold, wherein the prevascularized organoids are present in the hydrogel scaffold.

Administration in an embodiment of the methods is intramuscular. Administration may be intramuscular, intravenous, auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal.

In some embodiments, the organoids described herein may be used for screening agents that affect angiogenesis.

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The terms “isolated,” “purified,” or “biologically pure” refer to material that is free to varying degrees from components which normally accompany it as found in its native state.

The term “subject” as used in this application means a mammal. Mammals include canines, felines, rodents, bovine, equines, porcines, ovines, and primates including humans. Thus, the invention can be used in human medicine or also in veterinary medicine, e.g., to treat companion animals, farm animals, laboratory animals in zoological parks, and animals in the wild. The invention is particularly desirable for human medical applications. In a preferred embodiment the subject is a human.

The term “patient” as used in this application means a human subject. In some embodiments of the present invention, the “patient” is one suffering from an ischemic condition. In some embodiments of the present invention, the “patient” is one suffering from a vascular disease. In some embodiments of the present invention, the “patient” is one suffering from peripheral artery disease. In some embodiments of the present invention, the “patient” is one suffering from CLTI.

The terms “treat”, “treatment” of a disease, and the like refer to slowing down, relieving, ameliorating or alleviating at least one of the symptoms of the disease, or reversing the disease after its onset.

The terms “prevent”, “prevention”, and the like refer to acting prior to overt disease or disorder onset, to prevent the disease or disorder from developing or minimize the extent of the disease or disorder or slow its course of development.

The term “in need thereof” with regard to a subject would be a subject known or suspected of having or being at risk of developing an ischemic condition.

A subject in need of treatment would be one that has already developed an ischemic condition.

The terms “therapeutically effective amount” or “amount effective to” encompasses an amount sufficient to ameliorate or prevent a symptom or sign of the medical condition. Effective amount also means an amount sufficient to allow or facilitate diagnosis. An effective amount for a particular subject may vary depending on factors such as the condition being treated, the overall health of the patient, the method route and dose of administration and the severity of side effects. An effective amount can be the maximal dose or dosing protocol that avoids significant side effects or toxic effects.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Overview

Ischemic conditions, such as critical limb-threatening ischemia (CLTI), can result in amputation and death. Cell-based therapeutics, using endothelial cells or mesenchymal stem cells (MSCs), have shown potential to improve angiogenesis, but their clinical translation has been hampered due to immunogenicity of cells and low retention at the site of ischemia. By contrast, organoids present as larger multicellular aggregates with intriguing therapeutic potential, but have scale-up and production challenges. This work develops a strategy for treating ischemic conditions using prevascularized organoids. iPSC-derived endothelial cells (iECs) and human MSCs were combined in a sacrificial hydrogel multi-well template to form and release organoids. Endothelial differentiation and organoid sprouting capacity were characterized, and organoid function was tested and compared to single-cell equivalents in vitro and in vivo. Flow cytometry analysis showed over 95% EC purity. Organoids exhibited reproducible structures, with consistent diameters after 8 days of growth. Organoids showed a superior pro-angiogenic capacity in vitro and in the CAM assay, compared to single-cell equivalents. Importantly, organoid retention was significantly higher in the ischemic mouse hindlimb eight (8) days after injection compared to single-cell suspensions and enabled superior muscle regeneration. This work is a proof-of-principle that demonstrates that the prevascularized organoids are clinically translatable for therapeutic angiogenesis.

### Methods

Differentiation and Maintenance of iPSC-Derived Endothelial Cells (iECs)

Generation of iPSCs was carried out as previously described (42-44). For the generation of vascular endothelial cells (ECs) from iPSCs (iECs), a previous protocol (45) was optimized to make an “in-house” robust and efficient protocol. Briefly, iPSCs (OCT4 expression>90%) were plated in planar onto laminin plates as small colonies of cells, and 3 days later they were induced to mesoderm (ME—Phase I) using CHIR99021 (6 μM, Xcess Bio) for 2 days. Next, vascular progenitors (VP—Phase II) were generated using a combination of BMP4 (25 ng/ml, R&D Systems), FGF2 (10 ng/ml, PeproTech) and VEGF₁₆₅ (50 ng/ml, PeproTech) for another 2 days. The base medium used for Phases I and II was STEMdiff™APEL™2 medium (STEMCELL Technologies). On day 4 of differentiation, the majority of the VPs presented a cobblestone-like morphology in the periphery of the original iPSC colonies, VPs were dissociated with TrypLE (Sigma), and were re-plated in planar onto laminin-coated plates at a density of 10,000 cells/cm² in Endothelial Cell (EC) Growth Medium LV2 medium (also referred to as “GMPLV2 medium”) which is Endothelial Cell Basal Medium MV2 (PromoCell) supplemented with VEGF₁₆₅ (260 ng/ml), fetal bovine serum (0.05 mL/mL, Thermo Fisher Scientific), hydrocortisone (0.2 μg/mL) (STEMCELL Technologies), recombinant human FGF2-basic (153 a.a.) (10 ng/ml, PeproTech), animal-free recombinant human EGF, 6.2 kDa (5 ng/mL), L-ascorbic acid (1 μg/mL) and IGF-I LR3 (20 ng/mL, PeproTech). Cells were fed with this media to induce EC progenitors (ECP—Phase III) for 7 days. For purification and maturation of iECs (Phase IV), iECs were dissociated at Day 11 and re-plated at the same cell density onto laminin-coated plates with GMPLV2 media which was changed every other day for 10 days.

### MSC Maintenance

Adipose tissue-derived human MSCs (Promocell, Heidelberg, Germany) were cultured in Mesenchymal Stem Cell Growth Medium 2 (Promocell, Heidelberg, Germany). Cells were split at 80-90% confluency with Accutase (STEMCELL Technologies, Inc.) and used until passage 8.

### Immunocytochemistry Staining

96-well plate wells were coated with recombinant laminin in PBS (+/+Mg/Ca) for 2 hours at 37° C. followed by aspiration. iECs were seeded at a density of 30,000 cells per well with LV2 medium. When 80-90% confluent, cells were washed with PBS three times and then fixed with 4% paraformaldehyde (PFA) for 15 min. Cells were washed with PBS three more times and then blocked with PBS, 0.1% Triton-X (Sigma-Aldrich), and Donkey serum (Sigma-Aldrich) for 1 hour at room temperature. Blocking buffer was removed and primary antibodies were added and left overnight in a 4° C. fridge. Primary antibodies CD31 (PECAM-1) (89C2) Mouse mAb (1:100 dilution), CD144 (VE-Cadherin) Rabbit Antibody (1:200 dilution), VEGF Receptor 2 (55B11) Rabbit mAb (1:100 dilution) were purchased from Cell Signaling Technology, and vWF Polyclonal Rabbit Antibody was purchased from Invitrogen. The cells were washed with Tween diluted in PBS (1:1000) three times the next day. Then the cells were incubated with a 1:1000 dilution of secondary antibodies (Anti mouse IgG (H+L), F(ab′)2 Fragment (Alexa Fluor® 647 Conjugate, Anti rabbit IgG (H+L), F(ab′)2 Fragment (Alexa Fluor® 488 Conjugate), Cell Signaling Technology) for 1 hour at room temperature in the dark. The cells were washed with Tween four more times and then incubated with a 1:2500 dilution of DAPI in PBS for 15 min in the dark. Cells were washed with PBS three times and then imaged using fluorescence microscopy.

### Low Density Lipoprotein (LDL) Uptake Assay

iECs were seeded onto coated wells at 30,000 cells per well with LV2 medium. Dil-conjugated acetylated low-density lipoproteins (Dil-Ac-LDL) (Cell Applications, Inc.) were added in dark conditions to each well and incubated for 4 hours at 37° C. 5% CO2. DAPI was then added at a 1:2500 dilution in PBS for 15 min. Medium was then removed and cells were washed three times using a wash buffer, and examined using fluorescence microscopy.

### Flow Cytometry

Flow cytometry was performed on FACSCanto (BD Biosciences). Live/dead stain (Fixable Near-IR Dead Cell Stain Kit, 633 or 635 nm excitation) (Fisher Scientific), FITC-labeled mouse anti-human CD31 and Alexa Fluor 647-labeled mouse anti-human CD144 (BD Biosciences) were used. Briefly, iECs were lifted with TrypLE, washed with PBS once, and washed with buffer (10% FBS in PBS) twice. Antibodies were added and cells were washed twice more with buffer. The cells were then fixed with 4% PFA, washed once more with buffer, and then filtered through a 40 μm cell strainer (pluriSelect, Germany) before running flow cytometry.

### Organoid Formation

Organoids were formed according to a protocol developed in our lab (37). We formed organoids in 5 different cell combinations: only iECs, only MSCs, and 1:1, 1:3, and 1:9 iEC:MSC ratios. Briefly, we first split the cell types and stained them with cell tracking dyes (Cytotrace™ Red CMTPX for MSCs and Green CMFDA dye for iECs (Cayman Chemical)). Then we mixed the cell types into desired ratios and seeded them into sacrificial hydrogel scaffolds with microwells of 200 μm diameter. Each scaffold has ˜1,300 microwells, and approximately 600,000 K cells total were seeded onto each scaffold. These scaffolds were formed by placing 3% alginate onto a PDMS mold, and then adding 60 mM CaCl₂ for crosslinking. The mold was previously fabricated by casting PDMS into an SU-8 master mold. Scaffolds were then removed from the mold and placed into 24-well plate wells filled with 500 μL 1.8 mM CaCl₂. The 1.8 mM CaCl₂ was replaced with neutralization media (EC Basal Medium MV2+10% FBS) when cells were ready to be seeded into the scaffolds. On the day of seeding, iECs were D17 and hMSCs were P5-P8. The cells were left in the incubator for 30 min, after which 500 μL media was gently added to the wells. 500 μL of media was switched every two days, and GMPLV2 media was introduced on Day 4 of organoid growth. On Day 8, organoids were used in the CAM assay.

### Capillary-Like Vascular Sprout Analysis

The average number and length of sprouts were gathered for each organoid ratio by systematically noting presence or absence of sprouts in microscopy images of ˜160 organoids. Where a sprout was present, the total length was measured using ImageJ software. We defined capillary-like structures as any connection of iECs (stained green) that appeared to traverse through the middle of the organoid or project from the bulk of iECs within the organoid.

### Scratch Assay

iECs were grown on laminin-coated 24-well plates at a seeding density of 20,000 cells/well, until ˜90% confluence. Cell media was replaced with serum-free starvation media for 24 h. The cell layer was scraped using a 1000 μL pipette tip, after which cells were gently washed with PBS and replaced with media conditioned by 1:1 organoids, 1:1 single-cell equivalents or PBS. Light microscopy images were taken immediately after the scratch was induced (0 h), and 48 h after the scratch to assess migration of cells (Leica DMI6000 B).

### Tube Formation Assay

iECs were added to solidified growth factor reduced basement membrane matrix (Geltrex™ LDEV-Free, hESC-Qualified, Reduced Growth Factor Basement Membrane Matrix) in 96-well plates and cultured for 4 h with serum-free starvation media. After 4 h this media was replaced with 1:1 organoid-conditioned media, single cell-conditioned media conditioned or PBS. Microscopy images were taken at 24 h to assess the extent of tube formation of iECs caused by exposure to media (Leica DMI6000 B).

### Chick Chorioallantoic Membrane (CAM) Assay

The chick chorioallantoic (CAM) assay was performed using pathogen free fertilized eggs purchased from AVS Bio (catalog number 10100326). The eggs were incubated at 37° C. and 60-80% humidity. On embryonic development day 3 (ED3), the CAM for each egg was lowered by removing 3-5 mL of albumin using a needle inserted at the apex of the egg, and a small window ˜1 cm² was made in the shell. A piece of tape was applied to prevent falling of egg shells onto the CAM while cutting a window in the egg. The window was then resealed using a 3M™ Tegaderm™ transparent film dressing. On ED7, the Tegaderm™ was removed and a PTFE O-ring, (1 mm wide, 11 mm inner diameter) (McMaster-Carr) was placed on the CAM, imaged using a stereomicroscope (StereoMaster, Fisher Scientific), and 1:1 organoids, 1:1 single-cell equivalent suspensions, MSC-alone organoids, MSC-alone single-cell equivalent suspensions or PBS control was pipetted onto the CAM within the region defined by the O-ring. ˜300 K total cells were added in the case of single-cell suspensions and organoids, and 30 μL volume was used for single-cell suspensions, organoids and PBS controls. A total of n=3-4 healthy eggs were used for each treatment, a sample size in line with previous CAM assays (46) (47). Both types of organoids were made using the 200 μm scaffolds and grown until day 8. On embryonic development day 12, the CAM areas defined by the O-rings were imaged again and chick embryos were euthanized. Images were analyzed using ImageJ and AngioTool to quantify the extent of angiogenesis.

### Hindlimb Ischemia Surgery

Hindlimb ischemia was induced as previously described (48). Briefly, the femoral artery was ligated at a specific location which leads to arteriogenesis in femoral collaterals and angiogenesis in distal ischemic muscles. Mice were female, NU/J (homozygous), 12 weeks old and ˜20 g and were purchased from The Jackson Laboratory (strain 002019). After ischemia was induced, 300 K cells worth of iECs or organoids in saline were injected intramuscularly at a total volume of 24 μL (4×6 μL sites of injection, n=3-4 per group). iECs were labeled with fluorescent green CMFDA dye (Cayman Chemical). Equivoluminal injections of saline served as the control. Animals were housed separately and closely monitored. The surgeries were conducted with aseptic technique.

### Whole-Mouse Fluorescent Imaging

On days 1, 3, 8, 21 and 28 post-surgery, each mouse was imaged for fluorescence using (IVIS Spectrum In Vivo Imaging System; PerkinElmer; Waltham, MA). Aura software (Spectral Instruments Imaging) was then used to quantify fluorescence in the injection site, the hindlimb. Identically sized ROIs were chosen on the hindlimb, and a separate ROI was placed at random on the mouse body at each time point as “background”. The fluorescence was quantified as radiance by subtracting the background fluorescence from the fluorescence in the limb ROI.

### Functional Scoring Assessment

Mice were visually monitored without anesthesia on days 0, 1, 3, 7, 14, 17, 21 and 28 and assigned three functional scores to semi-quantitatively assess the functional impact of ischemia and any potential recovery after therapy. The Tarlov score, which describes the ability of the mouse to move, the ischemia score, which describes any necrosis, gangrene or pale foot abnormalities, and the modified ischemia score, which takes into account discoloration of nails, toes and amputation, were used (49).

### Histological Analysis

Mouse hindlimbs were fixed in 80% formalin for>24 hours before being placed in 70% ethanol. Pieces of hindlimb were then paraffin-embedded and sliced to 5 μm thickness. Slices were then stained for H&E or Masson's Trichome, before scanning at 40 ×on a Leica AT2.

### Statistical Analysis

One-way ANOVA with Tukey's multiple comparisons test was performed using Graphpad Prism 9 software.

### Results

### Generation of Organoids for Improving Angiogenesis

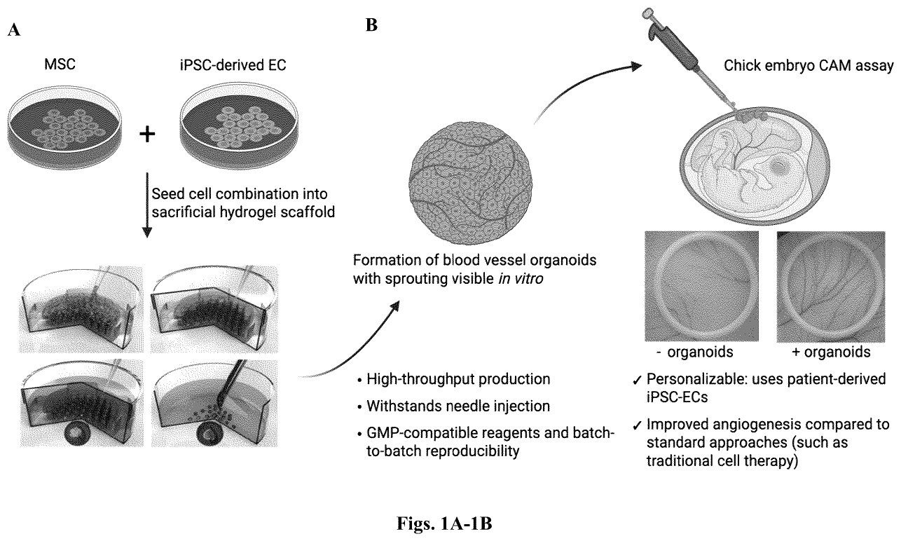

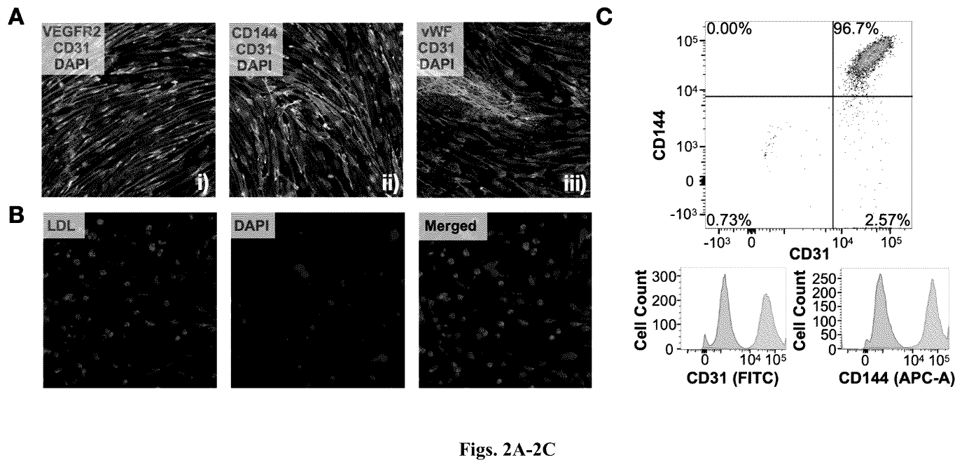

We developed injectable human prevascularized organoids as a therapeutic for increasing angiogenesis, which may be used, for example, in treating ischemic pathologies where revascularization is required. These organoids were fabricated by combining iPSC-derived endothelial cells (iECs) and human mesenchymal stem cells, adding them to our sacrificial hydrogel microwell scaffold platform, and allowing the cells to communicate and compact (FIGS. 1A-1B).

We leveraged our sacrificial hydrogel system wherein we deposit a sacrificial material (e.g., alginate), create the sacrificial microwell structure by cross-linking the alginate in its patterned state, and deposit cells atop the material to allow self-organization. This method allowed for production of a large number of organoids, independent of scaffold requirements, with controllable diameters and microstructures. In our design, we chose scaffolds with ˜1,300 microwells, such that ˜11,700 organoids could be produced per mold (for molds containing 9 scaffold templates). For human use, a therapeutic dose of 40 million MSCs as used in one CLTI clinical trial would only require just 8 molds to yield an equivalent amount of cells in organoids (at ˜600,000 cells per scaffold) (50). Moreover, we chose microwells with diameters of 200 μm, as organoids with diameters of ˜150 μm (after compaction) are below the diffusion limit of oxygen (200 μm), and contain more cells and potential therapeutic benefit than smaller organoids as shown previously (51).

Characterization of iECs and Organoids of Varying Cell Combinations

For clinical translation of cell-based therapies, it is important that the originating cells are scalable, defined and homogeneous. iECs were generated using a defined and stepwise protocol that is GMP-compatible. We have developed defined formulations for each step of iEC generation and culture (including our optimized iEC medium, GMPLV2). While animal-derived, a team from the FDA's Center for Biologics Evaluation and Research reviewed all regulatory filings for MSC products and found that over 80% of submissions mentioned the use of fetal bovine serum (FBS) during the cell therapy manufacturing process (52). The FDA has outlined that FBS may be used in the manufacture of cell products but evaluation of the source animal and collection procedures is required (53). As such, our protocol is GMP-compatible, should a GMP-grade FBS be used.

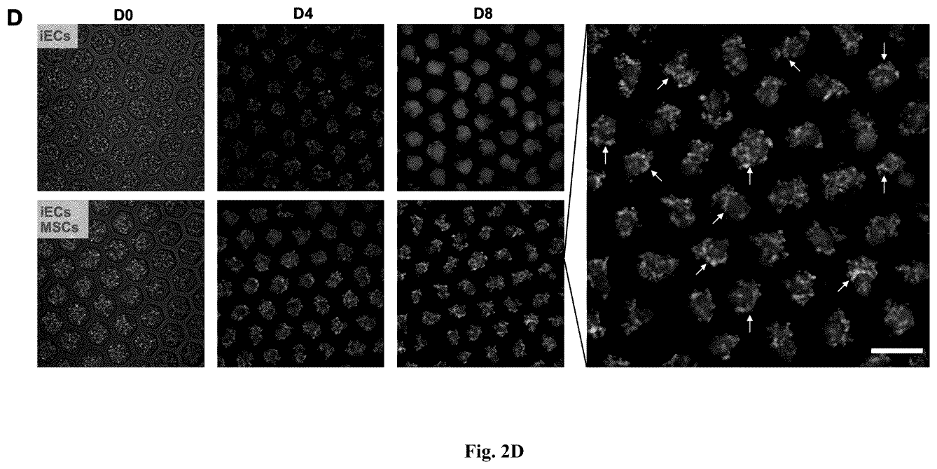

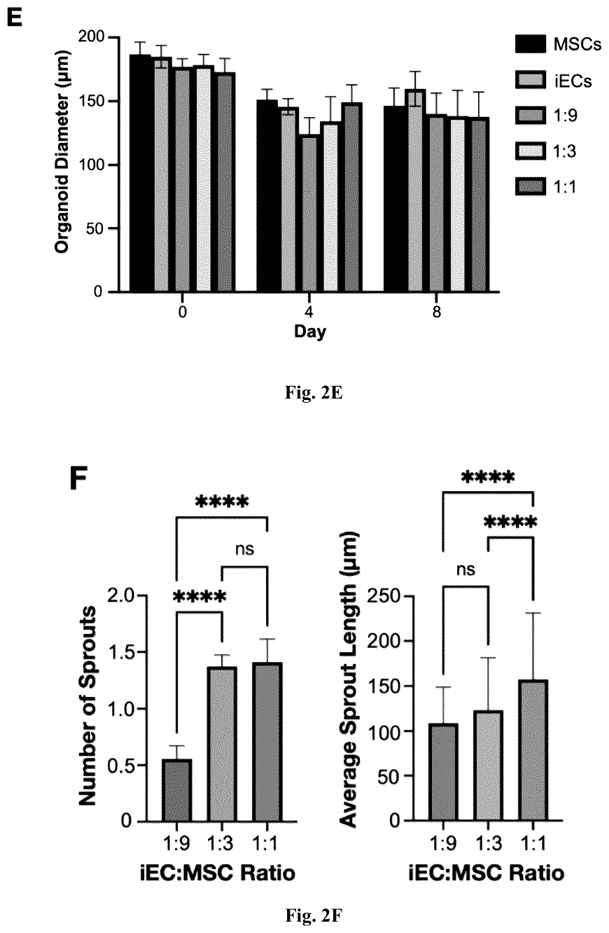

Immunocytochemistry for iECs was performed and confirmed that classical markers of ECs, including CD31, CD144, VEGFR2, and vWF, were present (FIG. 2A). The uptake of dil-conjugated acetylated low-density lipoproteins was also observed which confirms endothelial cell function (FIG. 2B) (54)(55). Flow cytometry showed that 96.7% of cells were positive for markers CD31 and CD144, demonstrating high purity (FIG. 2C). These data demonstrated the iECs were of high purity and contained the classic markers of endothelial cells.

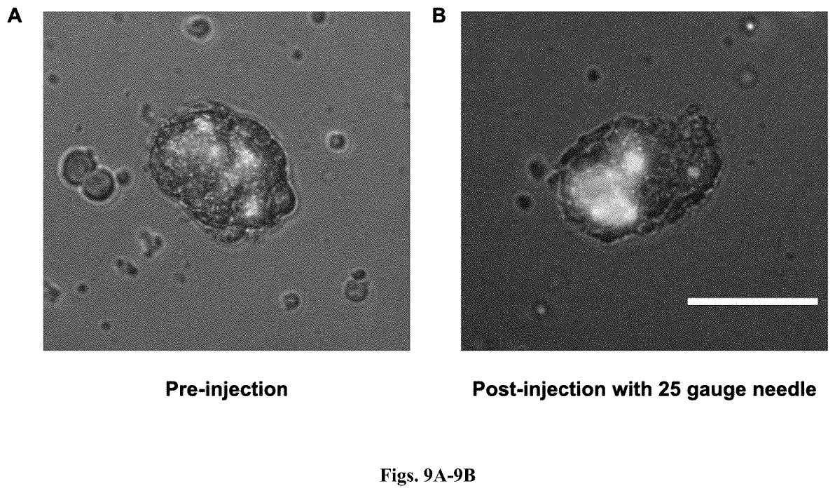

Next, iECs were deposited in sacrificial hydrogels to produce prevascularized organoids. Specifically, we placed different cell combinations into the microwells: iECs alone, MSCs alone, or iEC:MSC in the ratios of 1:1, 1:3, and 1:9. After 8 days of culture, the sacrificial layer was removed by adding a chelator (5% sodium citrate) to gently release the organoids, which were shown to be intact, without damage to structure or diameter (FIG. 9A). We also demonstrated the injectable nature of the organoids by injecting the organoids through 25-, 27- and 30-gauge needles and observing maintenance of organoid structural integrity and diameter after needle injection (FIG. 9B). We observed reproducible formation with consistent organoid diameters (FIG. 2D). Consistent with our previous observations (37), organoids compacted during the first four days and remained consistently sized through day 8 (denoted D8). At day 8, we observed no difference in diameter for all three iEC:MSC combinations—1:1, 1:3 and 1:9 (with diameters of 137.8±19.6 μm, 138.5±20.1 μm and 140.2±16.3 μm, respectively), further highlighting the ability to generate organoids containing different cell combinations to have reproducible diameters (FIG. 2E). The number of sprouts and sprout lengths were examined among organoids of different ratios, with the 1:1 iEC:MSC ratio showing the highest numbers in both measurements with on average 1.41±0.2 sprouts per organoid and 157.3±73.8 μm length (FIG. 2F). The higher degree of sprouting seen in the 1:1 ratio indicates that this construct may elicit improved angiogenesis compared to other ratios and thus was used for subsequent experiments.

### In Vitro Comparison of Organoid-Conditioned Medium and Single Cell-Conditioned Medium on Angiogenesis

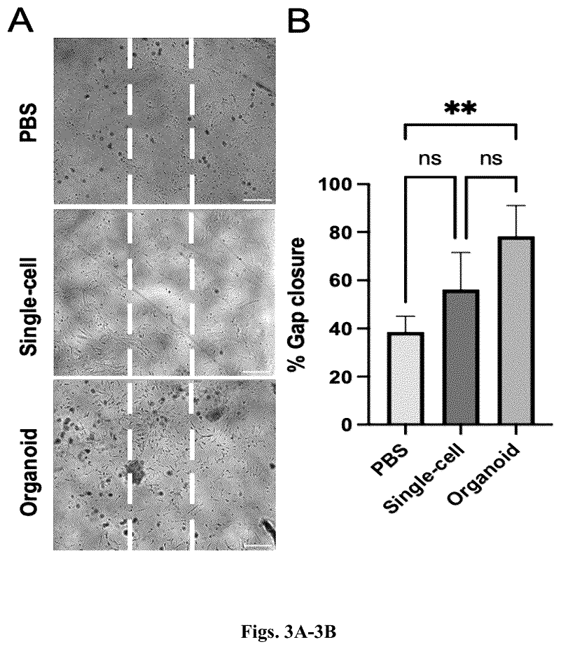

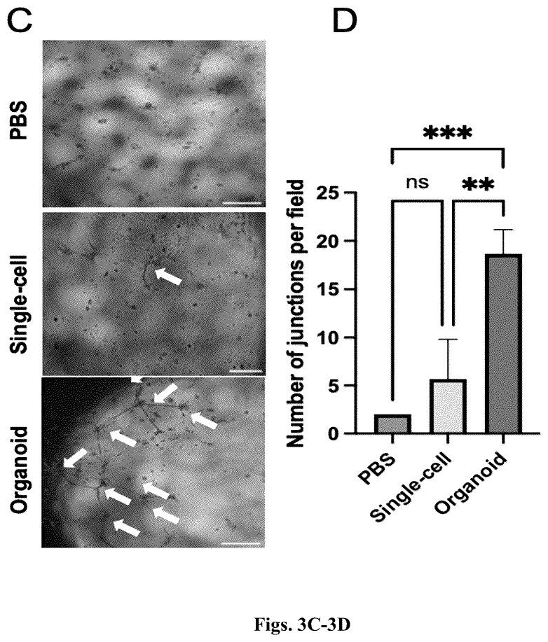

Since the assembly of MSCs and iECs into organoids resulted in capillary-like structures within the constructs, we investigated whether the iEC-MSC interactions and resultant secretions promote angiogenesis more than compared to their single-cell suspension equivalents. Endothelial cell migration is an essential step in angiogenesis (56). To evaluate this, iECs were treated with organoid-conditioned medium or single cell-conditioned medium. Secretions from both 1:1 iEC:MSC organoids and 1:1 iEC:MSC single-cell equivalent suspensions promoted migration of iECs after 48 h in a scratch assay (FIG. 3A and FIG. 3B). Wound areas closed to similar levels when iECs were exposed to organoid and single-cell secretions, and iECs migrated significantly more in response to organoid-conditioned medium compared to PBS control (p<0.005), consistent with previous results indicating that the secretome of EC:MSC organoids promote cell migration more so than single-cell equivalents (13). We also analyzed the effect of conditioned medium in a tube-formation assay using iECs. The formation of endothelial tubes on basement membrane matrix mimics many steps of angiogenesis (57). When treated with organoid-derived conditioned medium, significantly more stabilized tubular networks were formed by iECs on Geltrex after 24 hours (FIG. 3C and FIG. 3D) compared to single-cell conditioned medium (p<0.005) and PBS control (p<0.001). These results indicate that the iEC:MSC organoid secretome has potent angiogenic inductive ability, more so than the single-cell equivalent counterpart.

### In Vivo Studies of the Angiogenic Potential of Organoid Therapeutic in the Chick Chorioallantoic Membrane (CAM) Assay

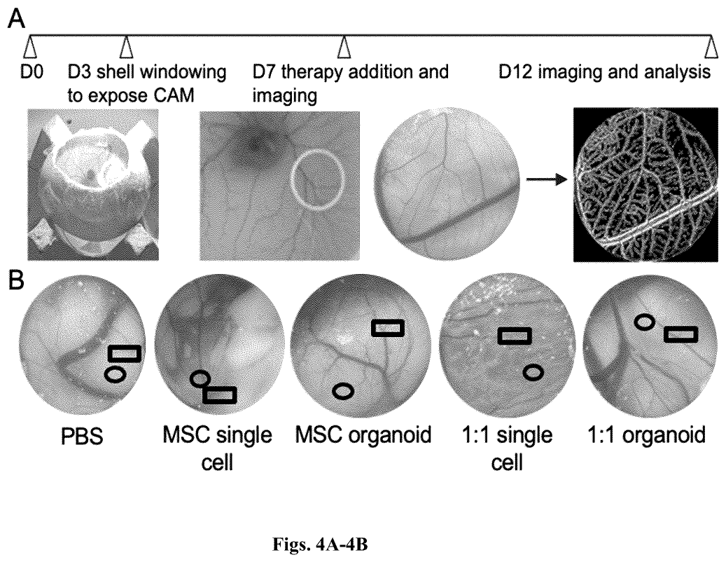

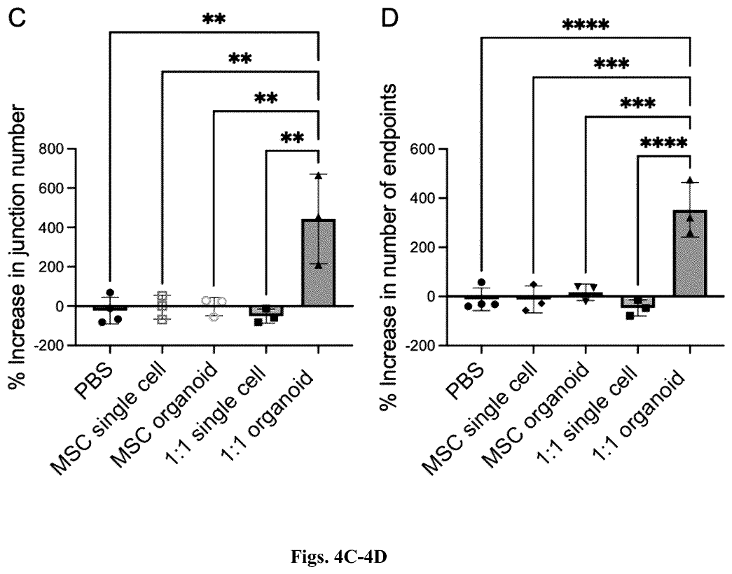

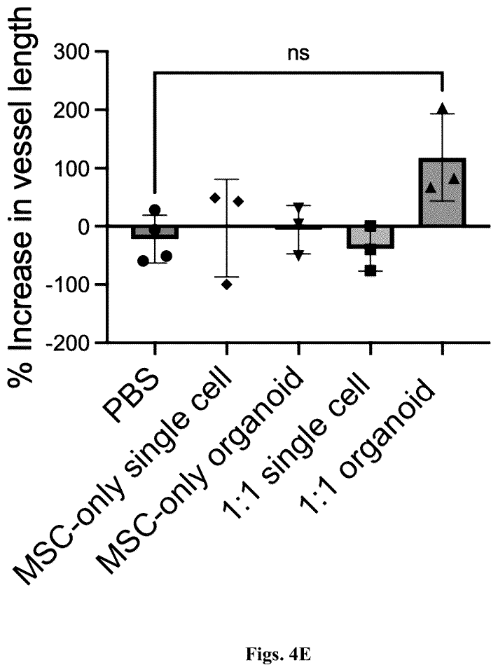

As an initial in vivo demonstration, we examined the effects of the iEC-containing organoids in a CAM assay, a validated angiogenic model in the chick embryo, to test the angiogenic effect of organoids compared to single-cell equivalent suspensions. 1:1 iEC:MSC organoids, 1:1 iEC:MSC single-cell equivalent suspensions of equal volumes (both containing 300,000 total cells), or PBS control of equal volume was added on top of the CAM on embryonic development day 7 (ED7). (A sample size of 3 to 4 healthy eggs were used for each treatment group, in line with previous CAM assays (46) (47). Treatments were added to a region of the CAM defined by a PTFE O-ring which was imaged immediately before addition of therapy, and again on ED12, five days after receiving therapy (FIG. 4A). Change in angiogenesis was measured individually for each embryo, since each embryo begins with a different baseline level of angiogenesis. The 1:1 iEC:MSC organoid treatments improved angiogenesis significantly more than single-cell equivalent, MSC-alone organoids, MSC-alone single-cell equivalents and PBS across multiple angiogenesis metrics. First, the 1:1 organoid treatment caused an increase in the number of vessel junctions of 443.38% by day 12, significantly more so than single-cell equivalent suspension, MSC-alone organoids and single-cell suspensions and PBS (p<0.005) (FIG. 4C). Second, the organoids caused an increase in the number of endpoints (i.e. open-ended segments, or tubes terminating in a growing tip (58) of 352.3% by day 12, significantly more so than single-cell equivalent suspension, MSC-alone organoids and single-cell suspensions and PBS (p<0.001) (FIG. 4D). Also, the 1:1 organoid treatment showed an increase in vessel length (FIG. 4E).

### Mouse Hindlimb Ischemia Study

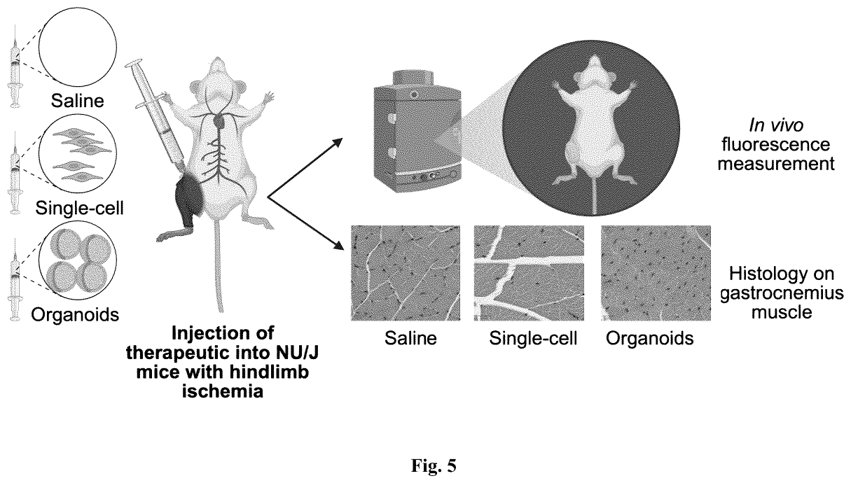

To uncover some mechanistic understanding of the organoids' superior abilities to increase angiogenesis in the CAM model, we next carried out a hindlimb ischemia mouse study. Briefly, 10 mice were subjected to femoral artery ligation as previously described (48). Each mouse received iEC-alone organoids, iEC-alone equivalent single-cell suspensions, or saline injection (FIG. 5) immediately after surgery induction. All iECs were tagged with Green CMFDA dye (Cayman Chemical). This allowed us to track organoid and single-cell fate within each mouse by using whole-body fluorescence imaging. We also performed laser speckle contrast imaging (LSCI), semi-quantitative functional scoring and histology on the gastrocnemius muscle.

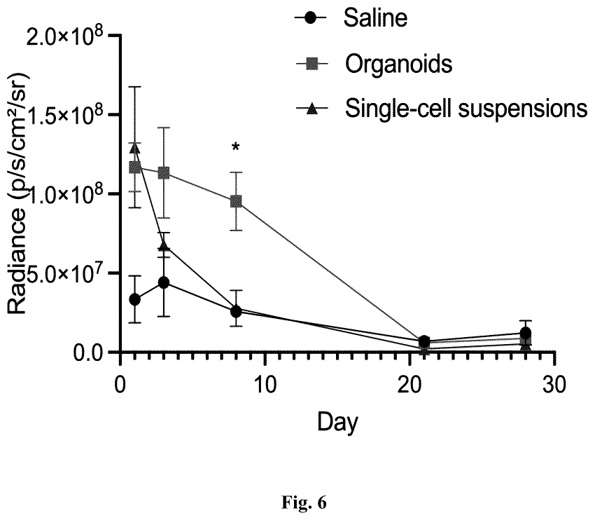

To this end, we hypothesized that injection of 3D iEC organoids may improve retention in the mouse hindlimb, which may explain superior efficacy of organoids compared to single-cell suspensions in the CAM model. We tracked fluorescence corresponding to Green CMFDA in the hindlimb on days 1,3,8,21 and 28 post-injection. Interestingly, by day 8 there was a statistically significant difference between fluorescence in mice receiving organoids compared to single-cell suspensions (FIG. 6). This observation indicates that organoids are retained more in the ischemic hindlimb compared to single-cell suspensions. Thus, the organoids may have more time to exert their therapeutic effects locally, and are not washed away rapidly as might be the case with single-cell suspensions. By day 21 there is little fluorescence visible in any group, which may be attributed to the short half-life of the fluorescent stain as opposed to using a genetically encoded fluorescent reporter. Fluorescent stains tend to be diluted with each round of cellular division (59). We have previously only used the fluorescent stain reliably for up to 8 days (for tracking organoid growth).

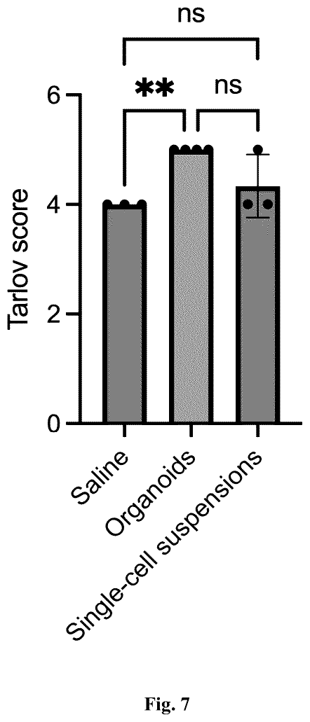

To track any functional improvements owing to either organoid or single-cell suspension therapy, the mice were assessed for three clinical grading scores commonly used in hindlimb ischemia: Tarlov score (assesses walking and ambulation), ischemia score (assesses the extent of gangrenous tissue on the ligated hind limb) and modified ischemia score (assesses the extent of necrosis using discoloration of nails) score (49). Interestingly, by Day 7, the organoid-treated mice had a statistically higher Tarlov score than saline-treated mice (FIG. 7) (p<0.01, Tukey's multiple comparison test after ANOVA). While there was no significant difference in organoid- vs. single-cell-treated mice, the trend indicated that organoid-treated mice recover faster than cell-treated mice.

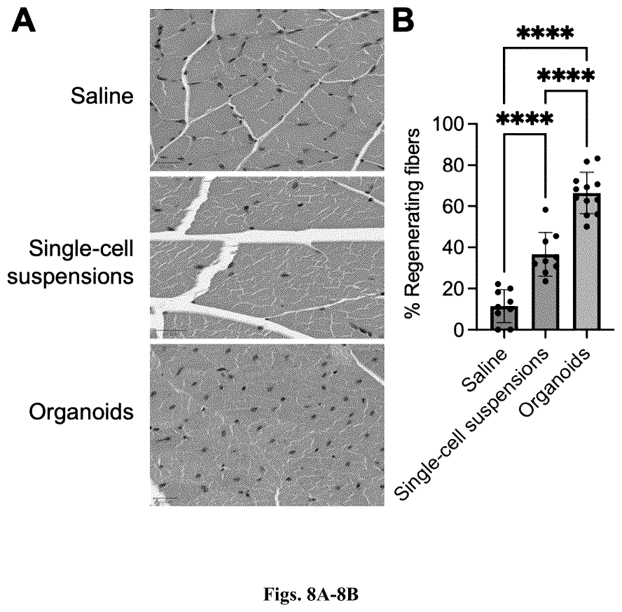

Finally, as a preliminary measure of the functional capacity of organoids or single-cell suspensions to promote regeneration, we performed hematoxylin and eosin (H&E) staining of the gastrocnemius muscle of each mouse 28 days after injury and injection with therapy. As a measure of muscle regeneration, we quantified the number of regenerating muscle fibers, identifiable by central nuclei in the fibers (60). Encouragingly, organoid-treated mice had a significantly higher fraction of regenerating fibers in the gastrocnemius muscle, compared to both single-cell suspensions and saline (FIGS. 8A-8B).

### Discussion

In this aim, we used the sacrificial hydrogel method (37) to generate prevascularized organoids containing a renewable and scalable source of vascular endothelial cells (namely, via iPSC technology with generation of iECs). Specifically, this study demonstrated the ability of the sacrificial hydrogel technique, which enables high-throughput production of organoids without requiring hydrogels such as Matrigel and which allows for gentle release, to successfully produce organoids containing iECs. The organoids were highly reproducible in size (138±20 μm for 1:1 iEC:MSC organoids by Day 8). Moreover, the organoids containing iECs exhibited sprouting, with the highest number of sprouts per organoid in the 1:1 iEC:MSC group (1.4±0.2 sprouts per organoid). Hence, this study demonstrates a clinically translatable manufacturing process for manufacturing prevascularized organoids containing clinically translatable cell sources, for use in, for example, revascularization treatment in ischemic diseases.

Endothelial sprouts or capillary-like structures have been noted to be in high abundance in organoids composed of endothelial colony-forming cells (ECFCs) and MSCs, whereas MSC-alone organoids demonstrate little to no sprout formation (61). Capillary-like structures have also been shown in EC: tumor cell organoids (62). In our study, the average number of sprouts and sprout length was highest in 1:1 iEC:MSC organoids, consistent with several previous studies indicating 1:1 EC:MSC is the ideal ratio (13) (63) (64). These studies have indicated that 1:1 EC:MSC organoids secrete the highest amount of paracrine factors such as VEGF and IGF-1, and exhibit the highest degree of EC network length and branching, shedding light on potential mechanisms contributing to their increased angiogenic potential.

Significantly, the results demonstrated that the prevascularized organoids improved angiogenesis compared to single-cell equivalent suspensions. These results were demonstrated both in in vitro assays and in the in vivo CAM assay. Organoid-conditioned media supported iEC tube formation significantly more than single cell-conditioned media and PBS, and promoted iEC migration in a scratch assay significantly more than PBS. In addition, 1:1 iEC:MSC organoids significantly improved angiogenesis in the CAM assay, more so than single cells and PBS, in terms of % increase in vessel length, number of junctions and number of endpoints. These results indicate that the secretome of 1:1 organoids is more pro-angiogenic than 1:1 single-cell suspensions.

The low retention rate of cells after direct injection into a hostile ischemic location such as the ischemic hindlimb has hampered clinical success (8). To overcome this, groups have 1) injected cells within hydrogel matrices including hyaluronan to promote retention and growth of transplanted cells (8) or 2) injected single cells in a 3D format such as spheroids. It was previously shown that MSC spheroids survived longer than single-cell MSC suspensions in a mouse model of hindlimb ischemia (12) and 3D spheroids of MSC/EC combinations survived longer than single-cell suspensions after transplantation in a mouse model of ischemic stroke, which led to superior efficacy (13). In our mouse hindlimb ischemia study, we showed that organoids are better retained in the injection site than single-cell suspensions at least up to day 8 post-injection. We also showed via histology that muscle regeneration was superior by 28 days post-surgery in the organoids-treated group. This may indicate that the enhanced retention afforded superior efficacy.

Towards clinical translation, the shift from preclinical to clinical manufacturing can be the most time-consuming and costly part of the process. Developing a GMP-compatible process early at a research phase ensures that once ready for large-scale GMP manufacturing, the process is compatible with regulatory compliance and scalability (mitigating risks related to critical starting raw materials, contamination, variability, or inconsistency). Here, we eliminated the need for Matrigel in developing vascular sprouts in the iECs, which suffers from lot-to-lot variation and carries a risk of zoonotic contamination. By contrast, use of defined, synthetic alternatives including defined serum alternatives, growth factors, small molecules, and extracellular growth matrices help to establish a consistent, reproducible, and safe process for generating iECs. We also developed a defined media incorporating GMP-compatible reagents for iEC differentiation. Although FBS is commonly used to generate cell therapeutics, its lot-to-lot variation has been criticized and has prompted the development of serum alternatives (65). We have also replaced FBS to a cGMP-grade human AB serum, placing the protocol even more in line with clinical translation.

This study demonstrates that organoids with pre-built vascular structures enable superior angiogenesis compared to single-cell treatment for conditions requiring revascularization procedures.

### Summary

Here, the production and in vivo effects of injectable prevascularized organoids composed of iECs and human mesenchymal stem cells (MSCs) was demonstrated using a high-throughput sacrificial alginate hydrogel system (37). Spheroids have been referred to as aggregated 3D structures created in a scaffold-free environment made from a broad range of cell types, and organoids as 3D structures generated from pluripotent stem cells and/or organ progenitor cells, among other features (38). As the structures in this study are composed of patient-derived endothelial cells, and exhibit capillary-like vascular sprouts rather than loosely aggregated cells, we refer to our structures as organoids. Previous reports of 3D cellular structures containing endothelial cells that exhibit capillary-like structures have also been referred to as organoids (31) (39). Towards translational use, hiPSCs were differentiated into high-purity (>90%) endothelial cells in a largely GMP-compatible manner by exploiting the Wnt signaling pathway and controlled addition of growth factors, eliminating the need for cell sorting. We assessed the organoid's ability to withstand needle injection and their angiogenic capacity in vitro, as well as an in vivo chorioallantoic membrane (CAM) assay, which has been useful for measuring the pro- or anti-angiogenic capacity of therapies (34,40,41). Further, we induced hindlimb ischemia in mice and compared the retention of cells and organoids, and assessed their potential to increase muscle regeneration.

### Bibliography

1. Shamaki GR, Markson F, Soji-Ayoade D, Agwuegbo CC, Bamgbose MO, Tamunoinemi B-M. Peripheral artery disease: A comprehensive updated review. Curr Probl Cardiol. 2022 November;47(11):101082.
2. Khaku AS, Tadi P. Cerebrovascular Disease. StatPearls. Treasure Island (FL): StatPearls Publishing; 2024.
3. Gulati R, Simari RD. Cell therapy for angiogenesis: embracing diversity. Circulation. 2005 Sep. 13;112(11):1522-4.
4. Ge Q, Zhang H, Hou J, Wan L, Cheng W, Wang X, et al. VEGF secreted by mesenchymal stem cells mediates the differentiation of endothelial progenitor cells into endothelial cells via paracrine mechanisms. Mol Med Report. 2018 January;17(1):1667-75.
5. Han MA, Jeon JH, Shin JY, Kim HJ, Lee JS, Seo CW, et al. Intramyocardial delivery of human cardiac stem cell spheroids with enhanced cell engraftment ability and cardiomyogenic potential for myocardial infarct repair. J Control Release. 2021 Aug. 10;336:499-509.
6. Cakir B, Xiang Y, Tanaka Y, Kural MH, Parent M, Kang Y-J, et al. Engineering of human brain organoids with a functional vascular-like system. Nat Methods. 2019 November;16(11):1169-75.
7. Pham MT, Pollock KM, Rose MD, Cary WA, Stewart HR, Zhou P, et al. Generation of human vascularized brain organoids. Neuroreport. 2018 May 2;29(7):588-93.
8. Tang ZCW, Liao W-Y, Tang ACL, Tsai S-J, Hsich PCH. The enhancement of endothelial cell therapy for angiogenesis in hindlimb ischemia using hyaluronan. Biomaterials. 2011 January;32(1):75-86.
9. Park JJ, Kwon YW, Kim JW, Park GT, Yoon JW, Kim YS, et al. Coadministration of endothelial and smooth muscle cells derived from human induced pluripotent stem cells as a therapy for critical limb ischemia. Stem Cells Transl Med. 2021 March;10(3):414-26.
10. MacAskill MG, Saif J, Condie A, Jansen MA, MacGillivray TJ, Tavares AAS, et al. Robust Revascularization in Models of Limb Ischemia Using a Clinically Translatable Human Stem Cell-Derived Endothelial Cell Product. Mol Ther. 2018 Jul. 5;26(7):1669-84.
11. Yao Z, Liu H, Yang M, Bai Y, Zhang B, Wang C, et al. Bone marrow mesenchymal stem cell-derived endothelial cells increase capillary density and accelerate angiogenesis in mouse hindlimb ischemia model. Stem Cell Res Ther. 2020 Jun. 8;11(1):221.
12. Song YC, Park GT, Moon HJ, Choi E-B, Lim M-J, Yoon JW, et al. Hybrid spheroids containing mesenchymal stem cells promote therapeutic angiogenesis by increasing engraftment of co-transplanted endothelial colony-forming cells in vivo. Stem Cell Res Ther. 2023 Aug. 2;14(1):193.
13. Hsu T-W, Lu Y-J, Lin Y-J, Huang Y-T, Hsich L-H, Wu B-H, et al. Transplantation of 3D MSC/HUVEC spheroids with neuroprotective and proangiogenic potentials ameliorates ischemic stroke brain injury. Biomaterials. 2021 May;272:120765.
14. Tanaka M, Tsuno NH, Fujii T, Todo T, Saito N, Takahashi K. Human umbilical vein endothelial cell vaccine therapy in patients with recurrent glioblastoma. Cancer Sci. 2013 February;104(2):200-5.
15. Kang SD, Carlon TA, Jantzen AE, Lin F-H, Ley MM, Allen JD, et al. Isolation of functional human endothelial cells from small volumes of umbilical cord blood. Ann Biomed Eng. 2013 October;41(10):2181-92.
16. Wylot B, Konarzewska K, Bugajski L, Piwocka K, Zawadzka M. Isolation of vascular endothelial cells from intact and injured murine brain cortex-technical issues and pitfalls in FACS analysis of the nervous tissue. Cytometry A. 2015 October;87(10):908-20.
17. Rowe RG, Daley GQ. Induced pluripotent stem cells in disease modelling and drug discovery. Nat Rev Genet. 2019 July;20(7):377-88.
18. Shi Y, Inoue H, Wu JC, Yamanaka S. Induced pluripotent stem cell technology: a decade of progress. Nat Rev Drug Discov. 2017 February;16(2):115-30.
19. Saha K, Jaenisch R. Technical challenges in using human induced pluripotent stem cells to model disease. Cell Stem Cell. 2009 Dec. 4;5(6):584-95.
20. Hamad S, Derichsweiler D, Gaspar JA, Brockmeier K, Hescheler J, Sachinidis A, et al. High-efficient serum-free differentiation of endothelial cells from human iPS cells. Stem Cell Res Ther. 2022 Jun. 11;13(1):251.
21. Wang L, Xiang M, Liu Y, Sun N, Lu M, Shi Y, et al. Human induced pluripotent stem cells derived endothelial cells mimicking vascular inflammatory response under flow. Biomicrofluidics. 2016 Jan. 13;10(1):014106.
22. Gu M. Efficient differentiation of human pluripotent stem cells to endothelial cells. Curr Protoc Hum Genet. 2018 Jul. 6;98(1):e64.
23. Hu X, White K, Olroyd AG, DeJesus R, Dominguez AA, Dowdle WE, et al. Hypoimmune induced pluripotent stem cells survive long term in fully immunocompetent, allogeneic rhesus macaques. Nat Biotechnol. 2024 March;42(3):413-23.
24. Kitano Y, Nishimura S, Kato TM, Ueda A, Takigawa K, Umekage M, et al. Generation of hypoimmunogenic induced pluripotent stem cells by CRISPR-Cas9 system and detailed evaluation for clinical application. Mol Ther Methods Clin Dev. 2022 Sep. 8;26:15-25.
25. Han X, Wang M, Duan S, Franco PJ, Kenty JH-R, Hedrick P, et al. Generation of hypoimmunogenic human pluripotent stem cells. Proc Natl Acad Sci USA. 2019 May 21;116(21):10441-6.
26. Hu X, Manner K, DeJesus R, White K, Gattis C, Ngo P, et al. Hypoimmune anti-CD19 chimeric antigen receptor T cells provide lasting tumor control in fully immunocompetent allogeneic humanized mice. Nat Commun. 2023 Apr. 10;14(1):2020.
27. Baghbaderani BA, Tian X, Neo BH, Burkall A, Dimezzo T, Sierra G, et al. cGMP-Manufactured Human Induced Pluripotent Stem Cells Are Available for Pre-clinical and Clinical Applications. Stem Cell Reports. 2015 Oct. 13;5(4):647-59.
28. Horiguchi I, Kino-oka M. Current developments in the stable production of human induced pluripotent stem cells. Engineering. 2021 February;7(2):144-52.
29. Haase A, Glienke W, Engels L, Göhring G, Esser R, Arseniev L, et al. GMP-compatible manufacturing of three iPS cell lines from human peripheral blood. Stem Cell Res. 2019 March;35:101394.
30. Sutherland RM, McCredie JA, Inch WR. Growth of multicell spheroids in tissue culture as a model of nodular carcinomas. J Natl Cancer Inst. 1971 January;46(1):113-20.
31. Markou M, Kouroupis D, Badounas F, Katsouras A, Kyrkou A, Fotsis T, et al. Tissue engineering using vascular organoids from human pluripotent stem cell derived mural cell phenotypes. Front Bioeng Biotechnol. 2020 Apr. 17;8:278.
32. Akiyama M, Nonomura H, Kamil SH, Ignotz RA. Periosteal cell pellet culture system: a new technique for bone engineering. Cell Transplant. 2006;15(6):521-32.
33. Wimmer RA, Leopoldi A, Aichinger M, Kerjaschki D, Penninger JM. Generation of blood vessel organoids from human pluripotent stem cells. Nat Protoc. 2019 November;14(11):3082-100.
34. De Moor L, Smet J, Plovyt M, Bekaert B, Vercruysse C, Asadian M, et al. Engineering microvasculature by 3D bioprinting of prevascularized spheroids in photo-crosslinkable gelatin. Biofabrication. 2021 Sep. 21;13(4).
35. Kozlowski MT, Crook CJ, Ku HT. Towards organoid culture without Matrigel. Commun Biol. 2021 Dec. 10;4(1):1387.
36. Park S, Cho S-W. Bioengineering toolkits for potentiating organoid therapeutics. Adv Drug Deliv Rev. 2024 Mar. 4;115238.
37. Rossen NS, Anandakumaran PN, Zur Nieden R, Lo K, Luo W, Park C, et al. Injectable therapeutic organoids using sacrificial hydrogels. iScience. 2020 May 22;23(5):101052.
38. Kang S-M, Kim D, Lec J-H, Takayama S, Park JY. Engineered microsystems for spheroid and organoid studies. Adv Healthc Mater. 2021 January;10(2):e2001284.
39. Wimmer RA, Leopoldi A, Aichinger M, Wick N, Hantusch B, Novatchkova M, et al. Human blood vessel organoids as a model of diabetic vasculopathy. Nature. 2019 Jan. 16;565(7740):505-10.
40. Dikici S, Aldemir Dikici B, MacNeil S, Claeyssens F. Decellularised extracellular matrix decorated PCL PolyHIPE scaffolds for enhanced cellular activity, integration and angiogenesis. Biomater Sci. 2021 Oct. 26;9(21):7297-310.
41. Di Marco GS, Alam A, Dol F, Corvol P, Gasc J-M, Larger E. Angiogenesis and diabetes: different responses to pro-angiogenic factors in the chorioallantoic membrane assay. Mol Med. 2008 Jul. 27;14(11-12):705-14.
42. Toombs J, Panther L, Ornelas L, Liu C, Gomez E, Martín-Ibáñez R, et al. Generation of twenty four induced pluripotent stem cell lines from twenty four members of the Lothian Birth Cohort 1936. Stem Cell Res. 2020 July;46:101851.
43. Baxi EG, Thompson T, Li J, Kaye JA, Lim RG, Wu J, et al. Answer ALS, a large-scale resource for sporadic and familial ALS combining clinical and multi-omics data from induced pluripotent cell lines. Nat Neurosci. 2022 Feb. 3;25(2):226-37.
44. Barrett R, Ornelas L, Yeager N, Mandefro B, Sahabian A, Lenacus L, et al. Reliable generation of induced pluripotent stem cells from human lymphoblastoid cell lines. Stem Cells Transl Med. 2014 December;3(12):1429-34.
45. Harding A, Cortez-Toledo E, Magner NL, Beegle JR, Coleal-Bergum DP, Hao D, et al. Highly Efficient Differentiation of Endothelial Cells from Pluripotent Stem Cells Requires the MAPK and the PI3K Pathways. Stem Cells. 2017 April;35(4):909-19.
46. Smith LM, Greenwood HE, Tyrrell WE, Edwards RS, de Santis V, Baark F, et al. The chicken chorioallantoic membrane as a low-cost, high-throughput model for cancer imaging. npj Imaging. 2023 Nov. 29;1(1):1.
47. Augustine R, Hasan A, Patan NK, Augustine A, Dalvi YB, Varghese R, et al. Titanium Nanorods Loaded PCL Meshes with Enhanced Blood Vessel Formation and Cell Migration for Wound Dressing Applications. Macromol Biosci. 2019 July;19(7):c1900058.
48. Limbourg A, Korff T, Napp LC, Schaper W, Drexler H, Limbourg FP. Evaluation of postnatal arteriogenesis and angiogenesis in a mouse model of hind-limb ischemia. Nat Protoc. 2009 Nov. 5;4(12):1737-46.
49. Brenes RA, Jadlowiec CC, Bear M, Hashim P, Protack CD, Li X, et al. Toward a mouse model of hind limb ischemia to test therapeutic angiogenesis. J Vasc Surg. 2012 December;56(6):1669-79; discussion 1679.
50. Mohamed SA, Howard L, McInerney V, Hayat A, Krawczyk J, Naughton S, et al. Autologous bone marrow mesenchymal stromal cell therapy for “no-option” critical limb ischemia is limited by karyotype abnormalities. Cytotherapy. 2020 June;22(6):313-21.
51. Cui L, Luo Y, Wang H, Chen Q, Zhou X, Guan Y, et al. Uniformly sized stem cell spheroids for treatment of hind limb ischemia: size effect. Adv Mater Interfaces. 2022 Mar. 25;2102327.
52. Mendicino M, Bailey AM, Wonnacott K, Puri RK, Bauer SR. MSC-based product characterization for clinical trials: an FDA perspective. Cell Stem Cell. 2014 Feb. 6;14(2):141-5.
53. Center for Veterinary Medicine FDA. CVM GFI #253 Current Good Manufacturing Practice for Animal Cells, Tissues, and Cell- and Tissue-Based Products. October 2022.
54. Azhdari M, Baghaban-Eslaminejad M, Baharvand H, Aghdami N. Therapeutic potential of human-induced pluripotent stem cell-derived endothelial cells in a bleomycin-induced scleroderma mouse model. Stem Cell Res. 2013 May;10(3):288-300.
55. Fan X, Cyganek L, Nitschke K, Uhlig S, Nuhn P, Bieback K, et al. Functional Characterization of Human Induced Pluripotent Stem Cell-Derived Endothelial Cells. Int J Mol Sci. 2022 Jul. 31;23(15).
56. Lamalice L, Le Boeuf F, Huot J. Endothelial cell migration during angiogenesis. Circ Res. 2007 Mar. 30;100(6):782-94.
57. Guo S, Lok J, Liu Y, Hayakawa K, Leung W, Xing C, et al. Assays to examine endothelial cell migration, tube formation, and gene expression profiles. Methods Mol Biol. 2014;1135:393-402.
58. Zhou H, Hong Y, Scoto M, Thomson A, Pead E, MacGillivray T, et al. Microvasculopathy in spinal muscular atrophy is driven by a reversible autonomous endothelial cell defect. J Clin Invest. 2022 Nov. 1;132(21).
59. Progatzky F, Dallman MJ, Lo Celso C. From seeing to believing: labelling strategies for in vivo cell-tracking experiments. Interface Focus. 2013 Jun. 6;3(3):20130001.
60. Narita S, Yorifuji H. Centrally nucleated fibers (CNFs) compensate the fragility of myofibers in mdx mouse. Neuroreport. 1999 Oct. 19;10(15):3233-5.
61. Shah S, Kang K-T. Two-Cell Spheroid Angiogenesis Assay System Using Both Endothelial Colony Forming Cells and Mesenchymal Stem Cells. Biomol Ther (Seoul). 2018 Sep. 1;26(5):474-80.
62. Shoval H, Karsch-Bluman A, Brill-Karniely Y, Stern T, Zamir G, Hubert A, et al. Tumor cells and their crosstalk with endothelial cells in 3D spheroids. Sci Rep. 2017 Sep. 5;7(1):10428.
63. Marshall J, Barnes A, Genever P. Analysis of the Intrinsic Self-Organising Properties of Mesenchymal Stromal Cells in Three-Dimensional Co-Culture Models with Endothelial Cells. Bioengineering (Basel). 2018 Oct. 26;5(4).
64. Carvalho DTO, Feijão T, Neves MI, da Silva RMP, Barrias CC. Directed self-assembly of spheroids into modular vascular beds for engineering large tissue constructs. Biofabrication. 2021 Apr. 2;13(3).
65. Karnieli O, Friedner OM, Allickson JG, Zhang N, Jung S, Fiorentini D, et al. A consensus introduction to serum replacements and serum-free media for cellular therapies. Cytotherapy. 2017 February;19(2):155-69.

1. A method for producing prevascularized organoids, the method comprising:
a) providing a sacrificial hydrogel scaffold;
b) seeding a population of cells comprising endothelial cells into the sacrificial hydrogel scaffold;
c) culturing the population of cells to form prevascularized organoids; and
d) removing the sacrificial hydrogel scaffold to release the prevascularized organoids.

2. (canceled)

3. (canceled)

4. (canceled)

5. The method of claim 1, wherein the hydrogel comprises about 3% alginate, wherein the sacrificial hydrogel scaffold is crosslinked,
wherein the sacrificial hydrogel scaffold comprises 500-2,000 microwells,
and/or wherein the sacrificial hydrogel scaffold comprises a plurality of microwells, wherein each microwell of the plurality is greater than 150 μm in diameter.

6. (canceled)

7. (canceled)

8. (canceled)

9. (canceled)

10. The method of claim 1, wherein about 1×10⁵ cells to about 5×10⁶ cells are seeded into the sacrificial hydrogel scaffold.

11. (canceled)

12. (canceled)

13. The method of claim 1, wherein the population of cells comprising endothelial cells comprises vascular endothelial cells, induced pluripotent stem cell (iPSC)-derived vascular endothelial cells (iECs), and/or mesenchymal stem cells (MSCs),
wherein the population of cells comprising endothelial cells comprises iECs and MSCs in an about 1:1 iEC:MSC ratio, and/or
wherein the population of cells is derived from an HLA-matched allogeneic source or an autologous source.

14. (canceled)

15. (canceled)

16. (canceled)

17. (canceled)

18. (canceled)

19. (canceled)

20. (canceled)

21. The method of claim 1, wherein the population of cells are cultured for about 8 days after seeding the cells into the sacrificial hydrogel scaffold;
wherein the sacrificial hydrogel scaffold is removed by exposing the sacrificial hydrogel scaffold to a chelator;
wherein the released prevascularized organoids comprise prevascularized organoids of about 120-160 μm in diameter, and/or wherein the released prevascularized organoids are injectable through a 25-gauge needle; and/or
wherein the method produces 500-10,000 organoids per scaffold.

22. (canceled)

23. (canceled)

24. (canceled)

25. (canceled)

26. (canceled)

27. (canceled)

28. (canceled)

29. An in vitro generated three-dimensional prevascularized organoid derived from a composition of cells comprising human iECs.

30. The prevascularized organoid of claim 29, wherein the iECs express CD31, CD144, VEGFR2, and vWF.

31. (canceled)

32. (canceled)

33. The prevascularized organoid of claim 29, wherein the composition of cells comprises iECs and MSCs in about 1:1 iEC:MSC ratio.

34. The prevascularized organoid of claim 29, wherein the organoid is about 120-160 μm in diameter, comprises about 1 to 2 vascular sprouts, and/or comprises a vascular sprout about 100-150 μm in length.

35. (canceled)

36. The prevascularized organoid of claim 29, wherein the human iECs are derived from an HLA-matched allogeneic source or an autologous source.

37. A composition comprising a plurality of the prevascularized organoids of claim 29, the plurality comprising between about 1,000 to 250,000 organoids.

38. (canceled)

39. (canceled)

40. A composition comprising the prevascularized organoids produced by the method of claim 1, secretions from the prevascularized organoids, and/or medium conditioned with the organoids.

41. The pharmaceutical composition comprising the plurality of prevascularized organoids of claim 29, and a physiologically acceptable or pharmaceutically acceptable carrier.

42. (canceled)

43. A method of treating a subject having, or at risk of having, an ischemic condition, the method comprising administering to the subject an amount of the composition of claim 37.

44. The method of claim 43, wherein the ischemic condition is critical limb-threatening ischemia (CLTI) and/or wherein the subject has or is at risk of having a vascular disease or peripheral artery disease (PAD).

45. (canceled)

46. (canceled)

47. (canceled)

48. A method of promoting angiogenesis in a subject, the method comprising administering to the subject an amount of the composition of claim 37.

49. (canceled)

50. (canceled)

51. (canceled)

52. (canceled)

53. The method of claim 48, wherein the composition or pharmaceutical composition is administered intramuscularly, into or proximal to ischemic tissue, and/or injected through a needle diameter greater than or equal to a 25-gauge needle;
wherein about 10,000 to 200,000 organoids are administered to the subject; and/or
wherein the administration 1 is an HLA-matched allogeneic administration or an autologous administration.

54. (canceled)

55. (canceled)

56. The method of claim 43, wherein about 10,000 to 200,000 organoids are administered to the subject.

57. The method of claim 43, wherein the administration is an HLA-matched allogeneic administration or an autologous administration.

58. The method of claim 43, wherein the subject displays increased angiogenesis, blood flow, tissue regeneration, and/or limb function at the site of administration.

59. (canceled)
